(19) United States
(12) Patent Application Publication
(10) Pub. No.: US 2022/0135924 A1
(43) Pub. Date: May 5, 2022

(54) METHOD AND DEVICE FOR CARRYING OUT REACTION PROCESSES

(71) Applicant: AQUILA BIOLABS GMBH, Baesweiler (DE)

(72) Inventors: Konrad Herzog, Herzogenrath (DE); David Frank, Herzogenrath (DE)

(21) Appl. No.: 17/431,614

(22) PCT Filed: Jan. 22, 2020
(86) PCT No.: PCT/EP2020/051455

§ 371 Date: Aug. 17, 2021

(30) Foreign Application Priority Data
Feb. 19, 2019 (DE) 10 2019 001 210.0

(51) Int. Cl. C12M 1/06; C12M 1/04; C12M 1/00; C12M 1/12; B01F 31/31; B01F 23/233

(52) U.S. Cl. CPC C12M 27/02; C12M 23/24; C12M 23/26; B01F 2101/44; C12M 29/20; B01F 31/31; B01F 23/233; C12M 37/02

(57) ABSTRACT

A method for carrying out a reaction process, in particular for setting the mixing and/or aeration of a reaction liquid while the reaction process is being carried out, which includes filling at least one reaction vessel with at least one reaction liquid, wherein an internal volume of the reaction vessel is not completely filled by the at least one reaction liquid at all times during the reaction process, and changing the internal volume of the reaction vessel in the course of the reaction process, in particular in a targeted manner, which causes a movement of the at least one reaction liquid.

### CROSS REFERENCE TO RELATED APPLICATIONS

This is a US national phase application under 35 U.S.C. § 371 of international application no. PCT/EP2020/051455, filed 22 Jan. 2020, which claims benefit of priority to German patent application no. 102019001210.0, filed 19 Feb. 2019; the entire content of each is herein incorporated by reference in its entirety

### TECHNICAL FIELD

The invention relates to a method for carrying out reaction processes and to a device for carrying out the method. Said invention can be used in particular for the cultivation of cells, as required, for example, in a wide variety of applications of high-throughput screening and development of media, strain or cell lines. The invention is advantageously applied in processes in which process-specific mixing or aeration rates have to be set.

### BACKGROUND OF THE INVENTION

Reaction processes can be found in all areas of research, development and production in the chemical, biological, biotechnological, biochemical and pharmaceutical industries. An important example of this is the cultivation of cells as an essential part of almost every bioprocess. To this end, reaction vessels are filled with culture medium and inoculated in a targeted manner with cells. The cells grow in the culture medium and are either themselves the target product of the bioprocess or they produce products such as enzymes or antibodies or act as biotransformation systems and thus as biological catalysts for the production of low-molecular-weight products from precursor molecules.

In order to achieve optimal process conditions, optimal conditions with regard to the mixing of the reaction liquid have to be set according to the reaction process. In order to achieve optimal growth conditions and production conditions, using the example of the cultivation of cells, optimal conditions with regard to the mixing and aeration of the reaction liquid have to be set according to the particular bioprocess, in particular according to the cell type used and the culture medium composition.

### PRIOR ART

A wide variety of methods and devices for carrying out reaction processes are known to a person skilled in the art These are explained in the following using the example process of the cultivation of cells, in which a reaction vessel is filled with culture liquid, the described methods and devices differing in particular with regard to their mixing and aeration of this culture liquid as the reaction liquid.

Shaken reaction vessels in which the reaction liquid is mixed by a shaking movement are known, in particular shake flasks, microtiter plates, reaction tubes and shaking bags. Usually, a plurality of reaction vessels are attached to a shaking platform and shaken together. The aeration of the reaction liquid takes place passively via gas-permeable closures, which close the openings of the reaction vessels. The gas exchange in the reaction vessel then takes place at the interface between the bulk of the reaction liquid and the gas phase in the headspace of the reaction vessel, as well as between said headspace and a liquid film, caused by the shaking movement, on the inner wall of the reaction vessel along the movement path of the reaction liquid. A drawback is that the movement of the reaction vessels and thus also the mixing of the reaction liquid contained in said vessels is the same for all reaction vessels shaken together, so that in most cases not every reaction process can be carried out under optimal conditions. Another drawback is that the aeration takes place passively, so that it is not possible to individually aerate the reaction liquid in a manner adjusted to the process requirements. Although devices in which aeration takes place actively via hoses and pumps are known from the prior art, this disadvantageously leads to a significantly more complex structure of the entire device with significantly more moving parts.

Also known are stirred reaction vessels, in particular stirred tank reactors, in which the reaction liquid is mixed by a rotating stirring device inside the reaction vessel. Such devices are actively aerated by bubble columns. In contrast to the shaken reaction vessels, stirred reaction vessels can be individually controlled both in terms of their mixing and in terms of the aeration of the reaction liquid, so that optimal process conditions can be set in this regard. A drawback, however, is the significantly higher complexity of the reaction vessels compared with shaken systems, which is in particular due to the necessary stirring components and aeration components in the interior of the reaction vessel. Another drawback is the significantly increased risk of foam formation due to the use of bubble columns compared with shaken reaction vessels, which can negatively affect the stability of products, catalysts and cells, as well as the general process conditions. The use of stirrers also has drawbacks: due to position-dependent mixing, which can lead to different reaction regimes in different regions of the reaction liquid; and also due to high shear forces which occur at the edges of the stirrer blades, and in cavitations caused thereby, and can damage sensitive cells, for example.

Flow reactors having bubble columns and pure bubble column reactors, in which the reaction liquid is mixed either by circulating pumping or by rising bubbles and the flow caused thereby, are also known to a person skilled in the art. A drawback of these systems is that they have a much more complex structure than the shaken reaction vessels due to the pumps and aeration components required, and, here too, zones of inhomogeneous mixing can arise, which have a detrimental effect on the success of the reaction process. As with all reaction vessels using bubble column aeration, there is also a significantly increased risk of foam formation, which can negatively affect the stability of products, catalysts and cells, as well as the general process conditions.

Methods and devices for resonance-acoustic mixing are also known (cf. Greta I. Reynoso-Cereceda et al., Shaken flasks by resonant acoustic mixing versus orbital mixing: Mass transfer coefficient kLa characterization and Escherichia coli cultures comparison, Biochemical Engineering Journal, vol. 105, 2016, pp. 379-390). A plurality of reaction vessels having the reaction liquid contained therein are moved vertically back and forth by an agitator platform, so that, due to the inertia of the reaction liquid, small droplets are detached and distributed in the headspace of the reaction vessels and thus high mixing and aeration rates can be achieved. It is advantageous here, analogously to the shaken systems, that a high degree of parallelization can be achieved with little effort and easily handleable reaction vessels. However, a drawback is that the setting of individual optimal process conditions with regard to mixing and aeration is not possible, exactly the same as in the case of the shaken systems. Another drawback is the strong atomization of the reaction liquid, which in some cases leads to an advantageous increase in the aeration rate of the reaction liquid, but which can also cause immense amounts of foam, which negatively affects the stability of products, catalysts and cells, as well as the general process conditions, and can also impede the mixing and aeration process itself since it interferes with the formation of droplets in the headspace. Disadvantageously, this foam can also reach the opening of the reaction vessel and either exit or close said vessel, so that gas transfer between the headspace and the ambient gas phase is no longer possible.

DE 4019182 A1 discloses a method for impregnating tissue samples in paraffin. An ultrasonic generator is arranged on a working vessel, by means of which generator the tissue sample is subjected to ultrasonic energy. The temperature of a fixing agent is intended to be increased by the ultrasonic energy of the ultrasonic generator.

Thus, no methods and devices are known that are suitable for carrying out reaction processes in a highly parallelizable manner under individually set, optimal conditions with regard to mixing and aeration, without having to resort to complexly constructed devices with invasive components or methods susceptible to foam formation.

### OBJECT OF THE INVENTION

It is therefore the object of the present invention to provide a method by means of which reaction processes can be individually set with regard to their mixing and, advantageously, with regard to their aeration, with at the same time high parallelizability, advantageously reduced or controllable susceptibility to foam formation and reduced complexity of the methods and devices to be used compared with the prior art

The method according to the invention is used in particular for the targeted setting of the mixing and/or aeration of a reaction liquid while reaction processes are being carried out.

### BRIEF SUMMARY OF THE INVENTION

According to the invention, the object is achieved by a method for carrying out reaction processes, which method is based on the basic concept of mixing the reaction mixture by means of a change, during the process, in the internal volume of the reaction vessel and the associated movement of the reaction liquid. The object is thus achieved according to the invention in particular by a method for carrying out reaction processes, in which method at least one reaction vessel is filled with at least one reaction liquid and the internal volume of the reaction vessel is not completely filled by at least one reaction liquid at all times during the reaction process, the method according to the invention being characterized in that the internal volume of the reaction vessel undergoes, in the course of the reaction process, a change which causes a movement of the at least one reaction liquid.

According to the invention, the change in the internal volume can also be accompanied by a change in the shape of the reaction vessel. In an advantageous embodiment of the invention, the method according to the invention can then be implemented in reaction vessels having at least one flexible region, for example by deforming, shifting or moving at least one flexible wall or at least one flexible region of at least one wall of the reaction vessel.

In one embodiment, the change in the internal volume is used in a targeted manner to set the mixing and/or aeration of the reaction liquid.

In one embodiment, the internal volume can be changed by 5%, preferably at least 10%, more preferably at least 20% or 50%.

In one embodiment, in addition to the reaction liquid, at least one headspace can also be provided in the internal volume, which headspace is in particular filled with a gas phase. According to the invention, the internal volume is changed such that the volume of the headspace is changed by 5%, preferably at least 10%, more preferably at least 20% or 50%.

In an advantageous embodiment of the invention, the changes in the internal volume or in the shape of the reaction vessel are repeated in the course of the reaction process, so that, for example, continuous periodic changes in the internal volume and the resulting movements of the reaction liquid allow continuous mixing of the reaction liquid throughout the entire reaction process.

According to the invention, the change in the internal volume or in the shape of the reaction vessel can be adjusted in the course of the reaction process, in particular, but not exclusively, with regard to its type, intensity, periodicity and speed. In an advantageous embodiment of the invention, the change in the internal volume or in the shape of the reaction vessel, and thus the mixing of the reaction liquid, is adjusted in response to the current reaction process state. According to the invention, suitable sensors for detecting and suitable models for mapping and describing this state provide, via a computer having suitable control software, the data necessary to adjust the changes in the internal volume of the reaction vessel.

In an advantageous embodiment of the invention, the change in the internal volume also allows active and controllable aeration of the reaction process by causing at least one gas transfer via at least one opening of the reaction vessel. According to the invention, gas flows in through at least one opening of the reaction vessel when the internal volume is increased, while gas is released from the reaction vessel through at least one opening when the internal volume is reduced.

In some embodiments of the invention, at least one opening is arranged in the reaction vessel in such a way that the gas transfer takes place between the headspace of the reaction vessel and the environment In other embodiments of the invention, the opening is arranged in the reaction vessel in such a way that the gas transfer takes place between the reaction liquid and the environment, such that increased aeration of the reaction liquid can be achieved due to the resulting bubble column.

In some embodiments, the gas transfer caused according to the invention by the change in the internal volume takes place through at least two openings of the reaction vessel, with the outflow of gas taking place via at least one opening which is in communication with the headspace, while the inflow of gas takes place via at least one other opening which is in communication with the reaction liquid.

In an advantageous embodiment of the invention, at least one opening of the reaction vessel is closed by means of a gas-permeable barrier such that liquid or solid substances cannot enter the interior of the reaction vessel or escape therefrom. In some embodiments of the invention, these barriers are designed as sterile barriers, in particular, but not exclusively, as sterile filters, cotton plugs or gas-permeable membranes.

In some embodiments of the invention, the size or shape of at least one opening of the reaction vessel can be adjusted automatically or manually in order to adjust the area available for the gas transfer to the requirements of the particular reaction process or reaction process state.

In an advantageous embodiment of the invention, the gas transfer between the headspace and the reaction liquid is increased by the change according to the invention in the size and shape of the internal volume in that, according to the invention, larger regions wetted with reaction liquid films are produced on the inner walls of the reaction vessel, which regions, due to the increased contact area with the gas phase of the headspace, allow an advantageously increased gas exchange.

The change according to the invention in the internal volume of the reaction vessel or its shape takes place depending on the embodiment of the invention by various movements of flexible walls of the reaction vessel, in particular, but not exclusively, by up-and-down or back-and-forth movements or by rolling, sliding or massaging movements.

In some embodiments of the invention, the reaction vessel comprises a flexible base which is moved up and down by an actuator. This actuator can in particular, but not exclusively, be designed as a permanent magnet or an electromagnet which is moved by an external magnetic field.

In some embodiments of the invention, the movement or deformation of at least one wall, which movement or deformation changes the internal volume, takes place completely actively by means of actuators. In other embodiments of the invention, only part of the movement is actively caused by at least one actuator, while the remaining part, in particular the return movement, is caused by the elasticity of the wall itself or spring elements attached thereto and acting thereon.

In some embodiments of the invention, at least one reaction vessel together with the reaction liquid contained therein is shaken during the process in order to achieve basic mixing, which is then individually adjusted, according to the requirements of the particular reaction process, using the method according to the invention.

In some embodiments of the invention, actuators which are suitable for deforming the reaction vessel are also used in order to generate flow obstacles that are adjustable in particular by deforming the base or the side walls of the reaction vessel, and that can set the turbulence and strength of the mixing.

According to the invention, the change in the internal volume or in the shape of the reaction vessel is actively carried out by one or more actuators. According to the invention, the change in the internal volume or in the shape of the reaction vessel can also be brought about passively by elasticity gradients or discrete elasticity differences in the walls of a shaken reaction vessel.

In some embodiments of the invention, the energy necessary to move the reaction liquid by changing the internal volume or the shape of the reaction vessel is monitored, analyzed and used in particular, but not exclusively, to detect changes in the mass or viscosity of the reaction liquid and to adjust the conditions of the reaction process accordingly.

In some embodiments of the invention, the device according to the invention in general, or the reaction vessel in particular, comprises suitable sensors in order to be able to detect the mixing, aeration and other essential process parameters of the process taking place in the reaction liquid and to be able to incorporate the process control. In particular, resistive or capacitive strain gauges can be attached to or in elastically deformable regions of the reaction vessel, which gauges allow the shape, distribution and mass of the reaction liquid to be assessed using the shape of the reaction vessel. In an advantageous embodiment of the invention, the change in the internal volume or in the shape of the reaction vessel in the context of the method according to the invention can thus be adjusted to its current requirements in the course of each reaction process.

### BRIEF DESCRIPTION OF THE DRAWINGS

FIG. 1 is a schematic representation of the method according to the invention.

FIG. 2 is a schematic representation of the method according to the invention with a change in the shape of the reaction vessel.

FIG. 3 is a schematic representation of the method according to the invention with gas transfer.

FIG. 4 is a schematic representation of a device according to the invention for carrying out the method according to the invention.

FIG. 5 is a schematic representation of a device according to the invention for carrying out the method according to the invention with a magnetic actuator.

### DETAILED DESCRIPTION

The present invention is explained in more detail with reference to the figures and embodiments. Reference signs in the figures designating components of the invention that have already been used in the same figure or in another figure under the same circumstances or in the same representation are partially omitted in order to maintain clarity of the figures. Graphic elements without reference signs are therefore to be interpreted in consideration of the list of reference signs, the other figures, the designated representations within the same figure, the patterning or structuring of already designated graphic elements and with reference to the entire description and the claims.

To ensure the clarity of some terms used in the description, these terms are defined and explained below and throughout the description.

A reaction vessel within the meaning of the invention is any device and any vessel that is suitable for receiving or storing reaction liquid. It can be open or closed. Reaction vessels within the meaning of the invention are therefore in particular, but not exclusively, shake flasks, reaction tubes, falcons, T-flasks, microtiter plates, shaking bags and shaking vessels of any geometry, material composition and filling capacity.

A reaction liquid within the meaning of the invention is a fluid in which at least one reaction relevant to the particular reaction process takes place. Reaction liquids within the meaning of the invention are therefore in particular, but not exclusively, culture broths, mixtures of culture medium and cells, chemical or biochemical reaction mixtures of solvents, starting materials, catalysts and products, and in principle all types of solutions, emulsions, dispersions, slurries, suspensions, foams, or powder mixtures with fluid properties.

Within the meaning of the invention, the headspace denotes the part of the internal volume of a reaction vessel which is not filled with reaction liquid. Said headspace is mostly filled with a gas phase and can therefore in particular, but not exclusively, contain air as well as any random or well-defined other gas mixture or pure gas, or can be completely or almost completely free of matter (vacuum).

Within the meaning of the invention, the internal volume denotes the inner volume of a reaction vessel that is enclosed by the reaction vessel. In the case of reaction vessels having at least one opening, the internal volume corresponds to the inner volume that would be enclosed if each existing opening were closed with the smallest possible area.

A change in the internal volume or in the shape of a reaction vessel is present within the meaning of the invention when the internal volumes or the shapes of a reaction vessel of at least two considered states of the reaction vessel or points in time of the reaction process are not identical.

Movements of the reaction liquid within the meaning of the invention are all movements that are suitable for mixing the reaction liquid or maintaining the mixing state of the reaction liquid.

Gas transfer within the meaning of the invention refers to any conceivable type of transport of at least one gas or gaseous molecule between two locations in space. Gas transfer within the meaning of the invention thus takes place in particular, but not exclusively, via diffusion, convection or via reactions such as evaporation, sublimation, condensation, solvation, desolvation, adsorption or desorption.

An actuator within the meaning of the invention is any device that is suitable for bringing about a change in the internal volume or in the shape of the reaction vessel. Actuators act in particular, but not exclusively, on flexible walls or wall components of the reaction vessel and on movably mounted components of the reaction vessel. Actuators within the meaning of the invention are in particular, but not exclusively, lifters, arms, pushers, slides, axes, eccentrics, permanent magnets, temperature-control elements, bimetal strips, hydraulic or pneumatic actuators, cranks, screws, liquids and piezo crystals.

A flexible wall or a flexible wall region within the meaning of the invention is any wall region of the reaction vessel that can be deformed, shifted or rotated by a suitable actuator. Flexible walls or flexible wall regions within the meaning of the invention are in particular wall regions made of flexible polymers, rubber, silicone, woven fabric, fleece, metal sheets, or foils. Flexible walls or flexible wall regions can have support structures or comprise optically transparent windows in order to allow optical sensors access to the reaction liquid in the reaction vessel. Flexible walls or flexible wall regions can contain or include, but in particular not exclusively, mechanical, capacitive, resistive, inductive, magnetic or optical sensors that allow characterization of the shape and distribution as well as other parameters of the reaction liquid or of the flexible wall itself

An actuator drive within the meaning of the invention is any device that is suitable for bringing about, conveying or adjusting the action of an actuator according to the invention. Actuator drives within the meaning of the invention are, in particular, motors, coils, electromagnets, pumps, heating and cooling elements, as well as voltage sources or current sources.

An actuator controller within the meaning of the invention is any device that is suitable for configuring, controlling or adjusting actuators or actuator drives according to the invention. Actuator controllers are often either analog control chains or computers, the latter including all devices, in particular electronic devices, that can store data (in particular arithmetic and logic data) and process said data on the basis of programmable rules. In particular, but not exclusively, microcontrollers, microprocessors, system-on-a-chip computers (SoC), PCs and servers, as well as networks of computers, are considered to be computers and thus also actuator controllers within the meaning of the invention.

A sterile barrier within the meaning of the invention is a gas-permeable device which is used in particular to prevent, reduce or completely stop the penetration of undesired cells, viruses or other contamination into the interior of the device according to the invention through at least one opening. Sterile barriers according to the invention allow at least one gas transfer between the headspace and the environment or the reaction liquid and the environment, in particular via diffusion or convection. Sterile barriers within the meaning of the invention are in particular, but not exclusively, sterile filters, porous membranes (e.g., PTFE, cellulose, hydrophilic or hydrophobic, etc.), cotton plugs or pads, and open-pore foams made of silicone, polyurethane or other plastics materials. Sterile barriers within the meaning of the invention are mostly connected to the wall of the reaction vessel, in particular, but not exclusively, by direct bonding or welding, as well as indirectly via suitable closure systems having a screw closure or locking closure or other interlocking or bonded connections.

A fastening device within the meaning of the invention is any device that is suitable for attaching at least one reaction vessel to another device, in particular, but not exclusively, to a table or a shaking platform. Fastening devices establish a mechanically loadable connection between at least one reaction vessel and at least one further device (in particular by means of an interlocking or bonded connection, negative pressure and by means of all types of gluing and adhesion).

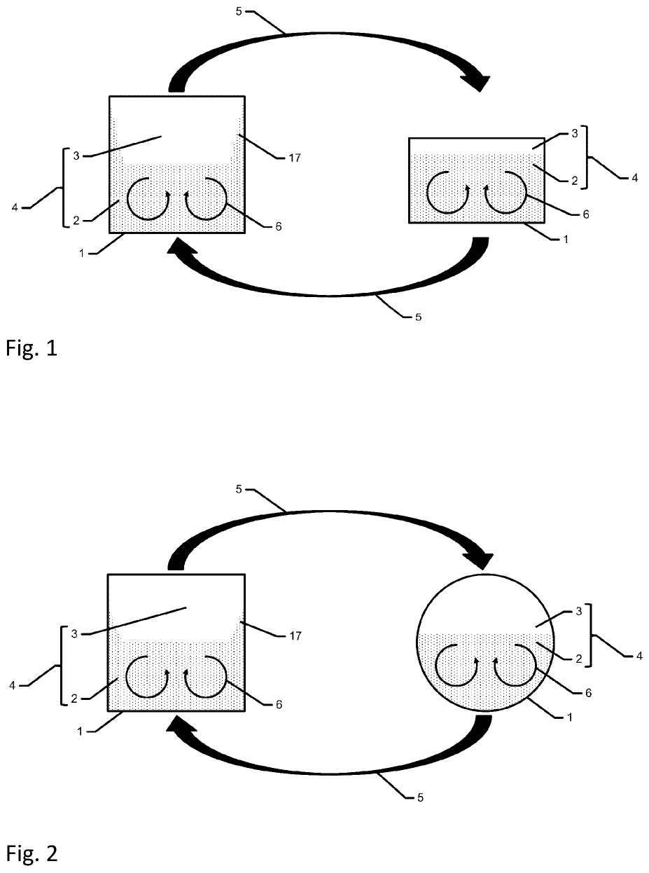

Turning now to the drawings, FIG. 1 is a schematic representation of the method according to the invention. For this, a reaction vessel 1 is filled with at least one reaction liquid 2 that is to be mixed in the course of the reaction process. However, the reaction liquid 2 does not fill the reaction vessel 1 completely at each point in time of the reaction process, but rather, in the reaction vessel 1, there is at least temporarily a headspace 3 in which there is no reaction liquid 2, but which is usually filled with a gas phase, in particular, but not exclusively, air. The entire internal volume 4 of the reaction vessel 1 thus comprises all regions of the reaction vessel 1 that are filled with reaction liquid 2 or that are part of the headspace 3. The method according to the invention is characterized by at least one change 5 in the internal volume 4 of the reaction vessel 1, the change 5 causing a movement 6 of the reaction liquid 2 and thus the mixing thereof.

In some embodiments of the invention, the internal volume 4 comprises, throughout the entire reaction process, at least one headspace 3, the gas phase of which can be compressed or decompressed by changes 5 according to the invention in the internal volume 4.

In an advantageous embodiment of the invention, as shown in FIG. 1, there are at least two different limit states of the internal volume 4, between which all internal volume changes 5 take place periodically continuously, such that the reaction liquid 2 performs movements 6 throughout the entire reaction process and is thus continuously mixed.

In an advantageous embodiment of the invention, the method according to the invention comprises the formation of a reaction liquid film 17 on the differential area of the different limit states of the internal volume 4. According to the invention, this reaction liquid film 17 is produced by the adhesion of the reaction liquid 2 to the inner walls of the reaction vessel 1. Said film can advantageously increase the gas exchange between the headspace 3 and the reaction liquid 2.

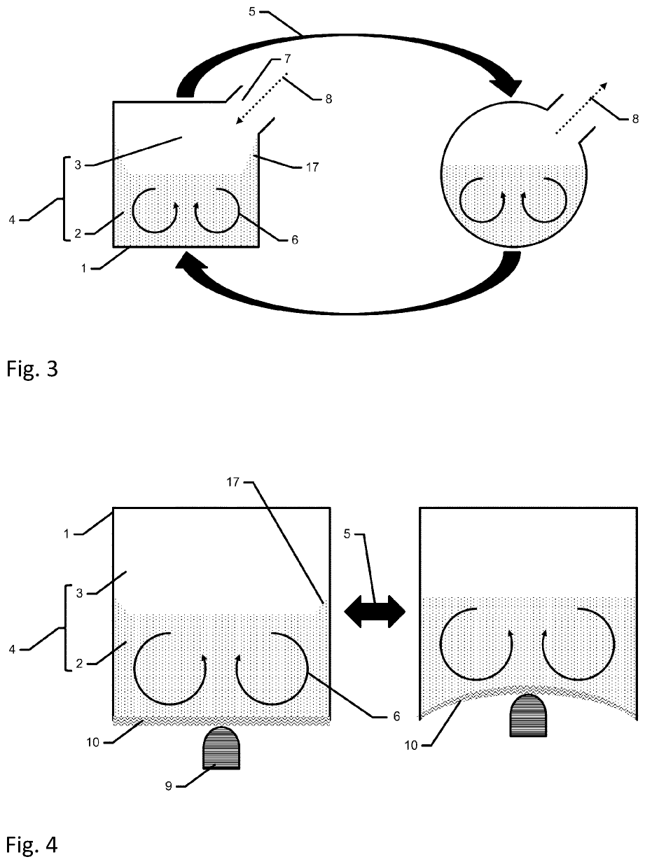

FIG. 2 is a schematic representation of the method according to the invention as in FIG. 1 but, in addition to the change 5 in the internal volume 4, also has a change in the shape of the reaction vessel 1. The movements 6 according to the invention of the reaction liquid 2 can thereby advantageously be intensified, or directed and influenced in a targeted manner. Thus, in an advantageous embodiment of the invention, the formation of inhomogeneously mixed regions in the reaction liquid 2 can be avoided by means of targeted deformation of the reaction vessel 1.

In an advantageous embodiment of the invention, the deformation of the reaction vessel 1 also takes place periodically continuously, so that the reaction liquid 2 performs movements 6 throughout the entire reaction process and is thus continuously mixed.

FIG. 3 is a schematic representation of the method according to the invention with deformation of the reaction vessel 1 as in FIG. 2, the reaction vessel 1 here also comprising an opening 7. According to the invention, changes 5 in the internal volume 4 of the reaction vessel 1 cause pressure changes in the headspace 3 which, as shown in FIG. 3, lead to a gas transfer 8 via the opening 7 of the reaction vessel 1.

According to the invention, therefore, increases in the internal volume 4 lead to gases flowing into the reaction vessel 1, and reductions in the internal volume 4 lead to gases flowing out of the reaction vessel 1. According to the invention, the intensity and speed of the gas transfer 8 and thus the aeration of the reaction liquid 2 can therefore be adjusted by adjusting the change 5 in the internal volume 4 of the reaction vessel 1, and controlled according to the requirements of the reaction process taking place therein.

The change in the internal volume can be considerable in order to achieve the desired aeration and/or mixing. A change in the internal volume by more than 5%, even more than 50%, is possible. It is also possible to change the volume of the headspace by this amount.

This type of aeration advantageously results in an oscillating aeration process similar to human breathing. The formation of foams is avoided here, but at the same time there is a significantly more efficient active gas exchange between the headspace 3 and the environment of the reaction vessel 1 compared with passively aerated shaking reactions. In addition, the division into the at least two shown states—with a large and a smaller headspace volume—advantageously results in a two-phase gas exchange process in which, in the inflow phase (left-hand side), a large surface area is available for gas exchange between the headspace 3 and the reaction liquid 2 due to the simultaneous formation, according to the invention, of a reaction liquid film 17, while the outflow phase (right-hand side) takes place using a gas mixture which, for example, has delivered oxygen to the reaction liquid 2 and has absorbed carbon dioxide from said liquid. Here too, the method according to the invention behaves similarly to human breathing.

FIG. 4 shows a schematic representation of a device according to the invention for carrying out the method according to the invention. The device comprises a reaction vessel 1 which is filled with a reaction liquid 2. The reaction vessel 1 furthermore comprises a headspace 3. This, together with the reaction liquid 2, fills the internal volume 4 of the reaction vessel 1. At least one wall or at least one part of at least one wall of the reaction vessel 1 is designed to be flexible, here in the form of a flexible wall 10. In order to carry out the method according to the invention, this flexible wall 10 is at least temporarily in contact with at least one actuator 9, by means of which the flexible wall 10 is shifted or deformed in order to bring about the change 5 according to the invention in the internal volume 4 or in the shape of the reaction vessel 1 and thus the movement 6 of the reaction liquid 2 necessary for the mixing.

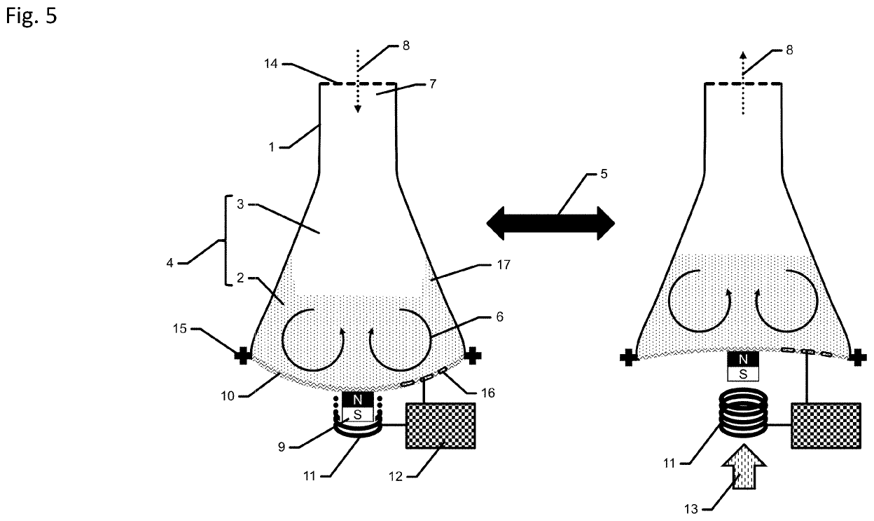

FIG. 5 is a schematic representation of a device according to the invention for carrying out the method according to the invention with a magnetic actuator 9. The reaction vessel 1 is designed based on an Erlenmeyer flask and is filled with reaction liquid 2. The design of the base of the reaction vessel 1 as a flexible wall makes it possible to carry out the method according to the invention. A permanent magnet is firmly connected to the flexible wall 10 as the actuator 9. This actuator 9 is moved up and down via a current-carrying coil, which is switchable and controllable by means of an actuator controller 12 and is in the form of an actuator drive 11, and the switchable external magnetic field 13 produced thereby. As a result, the shape and extent of the flexible wall 10 and thus the internal volume 4, and the shape of the reaction vessel 1, is changed according to the invention so as to produce a mixing movement 6 of the reaction liquid 2 and formation of the gas-exchange-promoting reaction liquid film 17.

The reaction vessel 1 shown also has an opening 7 through which, as already described for FIG. 3, the gas transfer 8 according to the invention between the headspace 3 and the environment of the reaction vessel 1 takes place. The opening 7 is closed by a gas-permeable sterile barrier 14 which prevents contamination of the reaction liquid 2.

Fastening devices 15 are also located on the reaction vessel 1, which devices allow the reaction vessel 1 to be attached to a shaking platform in order to achieve basic mixing of the reaction liquid 2 by means of shaking.

Furthermore, capacitive or resistive strain gauges 16 are located on or in the flexible wall 10 in order to detect the mass, shape and distribution of the reaction liquid 2 during the reaction process. These data can advantageously be integrated into the control and actuation of the actuator drive 11 via the actuator controller 12 and thus into the implementation of the method according to the invention.

In an advantageous embodiment of the invention with shaking operation, the actuator drive 11, the actuator controller 12 and the electrical circuits and computers necessary for detecting and analyzing the data from the strain gauges 16 are integrated into the shaking platform to which the reaction vessel 1 is attached by means of the fastening devices 15.

### LIST OF REFERENCE SIGNS

For the relevant interpretation of the reference signs, reference is made to the description and claims.

1 Reaction vessel

2 Reaction liquid

3 Headspace

4 Internal volume

5 Change in the internal volume or in the shape of the reaction vessel

6 Movement of the reaction liquid

7 Opening

8 Gas transfer

9 Actuator

10 Flexible wall or flexible wall region

11 Actuator drive

12 Actuator controller

13 External magnetic field

14 Sterile barrier

15 Fastening device

16 Strain gauges

1. A method for carrying out a reaction process, in particular for setting the mixing and/or aeration of a reaction liquid (2) while the reaction process is being carried out, the method comprising:
filling at least one reaction vessel (1) with at least one reaction liquid (2), wherein an internal volume (4) of the reaction vessel (1) is not completely filled by the at least one reaction liquid (2) at all times during the reaction process, and
changing (5) the internal volume (4) of the reaction vessel (1), in the course of the reaction process, in particular in a targeted manner, which causes a movement (6) of the at least one reaction liquid (2).

2. The method according to claim 1, characterized in that the change in the internal volume is used to set the mixing and/or aeration of the at least one reaction liquid (2) in a targeted manner.

3. The method according to either of the preceding claims, characterized in that the internal volume is changed by 5%, optionally at least 10%, or at least 20% or 50%.

4. The method according to claim 1, characterized in that in addition to the at least one reaction liquid (2), at least one headspace (3) is also provided in the internal volume, which is filled with a gas phase, and in that the internal volume is changed such that the volume of the at least one headspace is changed by 5%, optionally at least 10%, or at least 20% or 50%.

5. The method according to claim 1, characterized in that the change (5) in the internal volume (4) is accompanied by a change in shape of the reaction vessel (1).

6. The method according to claim 1, characterized in that the change (5) in the internal volume (4) or in shape of the reaction vessel (1) takes place repeatedly, periodically or continuously in the course of the reaction process.

7. The method according to claim 1, characterized in that the change (5) in the internal volume (4) causes at least one gas transfer (8) via at least one opening (7) of the reaction vessel (1).

8. The method according to claim 1, characterized in that at least one flexible wall (10) is deformed by at least one actuator (9) in order to achieve a change (5) in the internal volume (4) or in the shape of the reaction vessel (1) and thus to cause the at least one reaction liquid (2) to move (6) and to mix said at least one reaction liquid (2).

9. The method according to claim 1, characterized in that the change (5) in the internal volume (4) or in the shape of the reaction vessel (1) is adjusted to requirements of the reaction process in the course of said process.

10. A device for carrying out the method according to claim 1, comprising at least one reaction vessel (1) which contains at least one reaction liquid (2), wherein the internal volume (4) of the reaction vessel (1), at at least one point in time of the reaction process, also comprising, in addition to the at least one reaction liquid (2), at least one headspace (3), characterized in that
the reaction vessel comprises at least one flexible wall or at least one flexible wall region (10) and in that
the device comprises at least one actuator (9) which is designed, in particular for the targeted setting of the mixing and/or aeration of a reaction liquid, to bring about a change (5) in the internal volume (4) or in the shape of the reaction vessel (1).

11. The device according to claim 10, further comprising an actuator drive (11) and an actuator controller (12).

12. The device according to claim 10, characterized in that the reaction vessel (1) comprises at least one opening (7) which is suitable for bringing about at least one gas transfer (8).

13. The device according to claim 10, characterized in that at least one opening (7) of the reaction vessel (1) is closed by means of a sterile barrier (14).
